(19) United States
(12) Patent Application Publication
(10) Pub. No.: US 2026/0351375 A1
(43) Pub. Date: Nov. 5, 2026

(54) Perioperative Medication Control and Dispensation by Cobots

(71) Applicant: Peritas AI Inc., Fort Lauderdale, FL (US)

(72) Inventor: Martin Roche, Fort Lauderdale, FL (US)

(73) Assignee: Peritas AI Inc., Fort Lauderdale, FL (US)

(21) Appl. No.: 19/430,496

(22) Filed: Dec. 23, 2025

(51) Int. Cl. G16H 20/10; G06F 21/32; G16H 10/60; G16H 40/20; G16H 40/40; G16H 40/67

(52) U.S. Cl. CPC G16H 20/10; G06F 21/32; G16H 10/60; G16H 40/20; G16H 40/40; G16H 40/67

(57) ABSTRACT

A perioperative cobotic system is disclosed for secure, context-aware medication dispensation in surgical environments. The system comprises a mobile or semi-stationary collaborative robot equipped with an access-controlled medication storage module, a multi-factor clinician authentication interface, and an integrated control unit. The control unit interfaces with electronic medical records and surgical scheduling systems to authorize dispensation based on patient identity, surgical stage, and clinician credentials. The system further includes a context-aware inference engine that evaluates patient vital signs, procedural cues, and OR alerts to anticipate medication needs and support real-time recommendations. A user interface supports both touchscreen and voice interaction for medication requests and confirmations. Real-time event logging and wireless integration with hospital systems enable inventory updates, audit trails, and EMR synchronization. The cobot navigates autonomously within the OR while employing sterile field detection and obstacle avoidance to ensure safe and compliant operation.

### CROSS-REFERENCE TO RELATED APPLICATIONS

This patent application is a continuation-in-part of US Patent Application Ser. No. 19/195,731 filed on Apr. 30, 2025, which claims the benefit of priority to U.S. Provisional Patent Application No. 63/640,524, filed on Apr. 30, 2024, the entirety of which is incorporated herein by reference.

### TECHNICAL FIELD

The present invention relates generally to robotic systems and hospital automation, and more particularly to collaborative robotic platforms configured for secure, context-aware medication control and dispensation within perioperative clinical environments.

### BACKGROUND

Modern surgical workflows increasingly rely on technology to streamline operations, enhance safety, and reduce clinical burden. However, the dispensation and administration of medications in perioperative settings remain highly manual, dependent on clinical personnel to retrieve, verify, and deliver medications at critical surgical junctures. This process is vulnerable to delays, human error, and documentation inconsistencies, particularly in high-acuity environments where timing and accuracy are essential.

Conventional medication dispensing systems are typically located outside the sterile field, requiring nurses or anesthesiologists to leave their immediate stations to access automated dispensing cabinets or pharmacy stockrooms. These interruptions not only consume valuable time but may also compromise situational awareness during active procedures. Furthermore, existing systems often lack fine-grained control over dosage-level dispensing, do not consistently enforce multi-factor clinician verification, and are not tightly integrated with surgical scheduling data or electronic medical records (EMRs) to enforce contextual dispensation rules.

Additional limitations stem from the absence of real-time auditability and safety compliance. For example, current systems do not maintain immutable logs of medication events synchronized to the surgical timeline, nor do they autonomously monitor for sterile field breaches during dispensation activities. As perioperative environments become increasingly dynamic, there is a critical need for intelligent, secure, and context-aware medication delivery systems that can operate autonomously or collaboratively within the operating room.

Accordingly, there is a need for a cobotic platform that integrates secure medication storage, EMR-linked control logic, real-time clinician authentication, and autonomous dispensation capabilities. Such a system would enhance the speed, accuracy, and traceability of perioperative medication delivery while maintaining compliance with sterile field protocols and hospital pharmacy regulations.

### SUMMARY

The present invention relates to a perioperative cobotic platform configured for autonomous or semi-autonomous medication transport and dispensation within surgical environments. The system comprises a mobile or semi-stationary collaborative robot equipped with a secure, access-controlled medication storage module, capable of dispensing various medication types with dosage-level precision. Integrated with hospital systems via a wireless communication interface, the cobot validates dispensing requests against surgical schedules and electronic medical records (EMRs), confirming patient identity, surgical stage, and prescriber authorization.

Prior to dispensing, the system enforces clinician authentication through at least two verification methods, including biometric identification (e.g., facial or fingerprint recognition), badge scanning, or PIN entry. Context-aware dispensation logic evaluates procedural cues, such as surgical phase, vital signs, or operating room alerts, to anticipate or recommend medication delivery. A clinician-facing user interface, accessible via touchscreen or voice control, allows for confirmation, override, or cancellation of dispensing actions during surgery.

Each dispensation event is automatically logged with detailed metadata, including clinician identity, medication type, dosage, time, and location. Real-time updates are transmitted to pharmacy, inventory, and patient management systems, enabling dynamic inventory control and auditability. Integrated safety protocols ensure sterile field compliance, obstacle avoidance, and workflow continuity, allowing the cobot to function safely and reliably in active perioperative zones.

### BRIEF DESCRIPTION OF DRAWINGS

The foregoing and other objects, aspects, features, and advantages of the disclosure will become more apparent and better understood by referring to the following description taken in conjunction with the accompanying drawings, in which:

FIG. 1 is a perspective view of a cobotic medication dispensation platform.

FIG. 2 is a block diagram of the system architecture.

FIG. 3 is a flowchart illustrating the multi-factor clinician authentication and verification process prior to dispensing.

FIG. 4 is a schematic diagram of the context-aware dispensation logic.

FIG. 5 is a workflow diagram showing autonomous dispensation authorization, event logging, and data synchronization with hospital systems.

FIG. 6 is a top-down schematic of cobot positioning and obstacle avoidance in an operating room.

FIG. 7 is a user interface screen layout.

FIG. 8 is a diagram illustrating system integration with hospital inventory and pharmacy databases.

These drawings are provided for illustrative purposes only and should not be construed as limiting the invention to the specific embodiments shown and described herein. Variations and modifications to these embodiments will be apparent to those skilled in the art, and all such variations and modifications are intended to be included within the scope of this disclosure.

### DETAILED DESCRIPTION

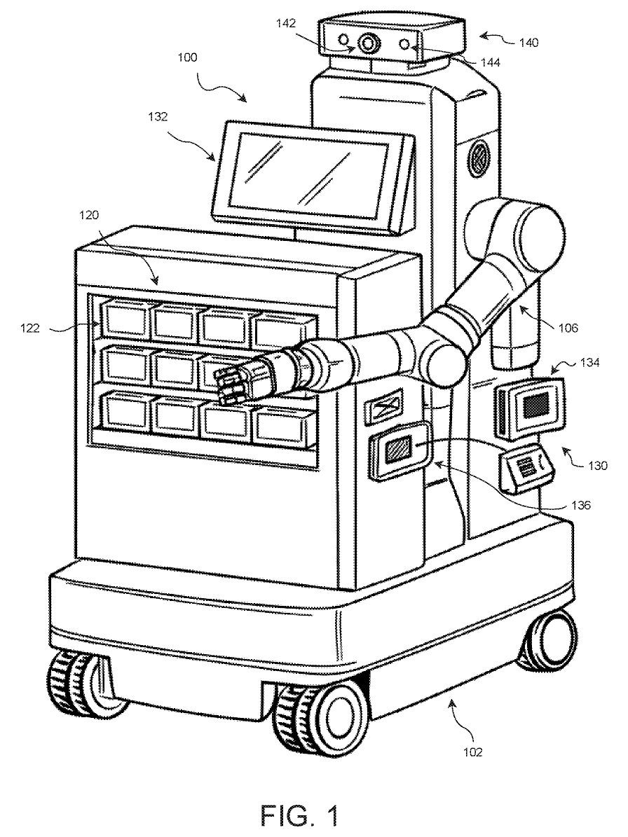

FIG. 1 illustrates an example perioperative medication dispensing cobot system 100 configured for use in a surgical environment. The cobot 100 includes a mobile base 102 supporting a secure medication storage module 120, and a human-machine interface (HMI) 130, and various sensors 140. In one embodiment (the “humanoid” cobot), the system further comprises one or more articulated robotic limbs 106 mounted on the mobile base 102. These limbs 106 can manipulate medication containers and interact with the environment, while the base 102 provides autonomous mobility to reposition the cobot within an operating room (OR). In an alternate embodiment, the cobot is implemented as a wheeled dispensing unit without articulated arms (a “wheeled-only” version). In this wheeled-only design, the cobot 100 still contains the secure storage module 120 and HMI 130 on a mobile base, but medications are accessed directly from the base’s compartments by clinical staff rather than being handed over by a robotic arm. Both embodiments share the core features of secure medication storage, controlled dispensing, clinician authentication, and context-aware operation, differing primarily in how medications are delivered to the user (via robotic arm in the humanoid version versus direct compartment access in the wheeled-only version).

The secure medication storage module 120 is an integrated, tamper-resistant cabinet on the cobot that houses multiple compartmentalized storage units (e.g., drawers or bins 122). Each compartment 122 holds a specific medication type or dose (for example, unit-dose vials of anesthetics, antibiotic syringes, or emergency drugs) and remains locked until an authorized dispense command is given. The compartments are individually controlled so that only the intended medication’s compartment opens for a dispense event, helping to prevent unauthorized access or selection errors. This design is similar to automated dispensing cabinet (ADC) technology in that it allows medications to be stored at the point of care with controlled access and tracking of drug distribution. The storage module 120 may include sensors to detect inventory levels (such as weight sensors or RFID tag readers in each compartment) and to confirm when an item has been removed. Dosage-level precision is enabled by stocking pre-measured unit doses or by mechanisms that dispense exact quantities (for instance, pill counting mechanisms or syringe filling interfaces), ensuring that each dispense action provides an accurate amount of medication. The module 120 is constructed with appropriate materials and locking features to maintain medication security and environmental controls (e.g., climate control for temperature-sensitive drugs, if needed).

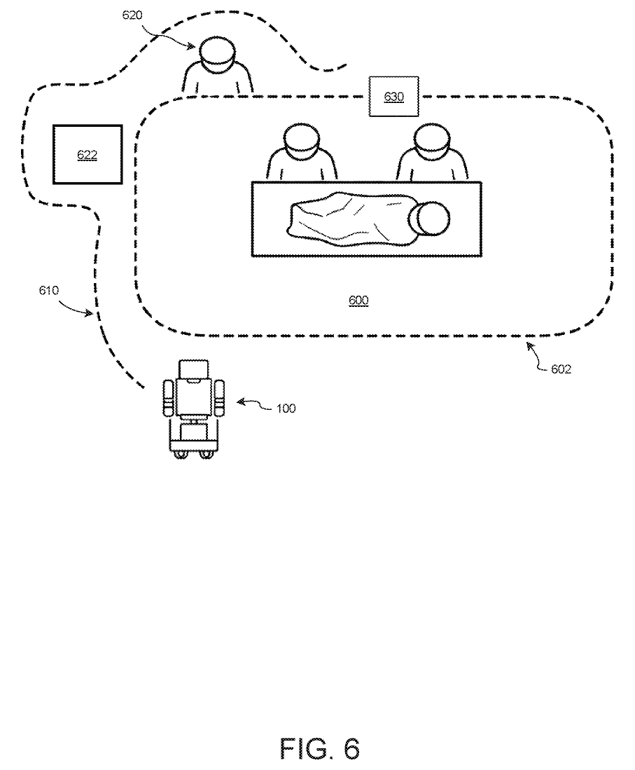

Mounted on the cobot is a multi-modal clinician authentication interface and HMI 130. In the depicted embodiment, a touchscreen display 132 is provided for user interaction, along with integrated hardware for multi-factor authentication: for example, a fingerprint scanner 134, an RFID/badge reader 136, and a numeric keypad (which may be implemented on the touchscreen for personal identification number (PIN) entry). The cobot also incorporates audio/visual indicators and interfaces, including a speaker and microphone (as part of HMI 130) to enable voice interaction and to provide spoken alerts or confirmations to staff. One or more cameras 142 and depth sensors are positioned on the cobot (for instance, in the head or mast of a humanoid form, or on the chassis of a wheeled form) to facilitate navigation, obstacle detection, and monitoring of the surroundings. These sensors 140, along with others such as ultrasonic proximity sensors or LiDAR units 144 around the base, enable the cobot to perceive its environment in 360 degrees for safe movement. In addition, the cobot 100 is equipped with specialized sensors or software for sterile field detection, for example, machine vision algorithms that recognize surgical drapes or use depth sensing to map out the sterile zone around the patient. The system can thus define a virtual boundary representing the sterile field and monitor its own position relative to that boundary in real time. Any encroachment or risk of contaminating the sterile field can be detected by these sensors and avoided through the cobot’s control system (as described later with respect to FIG. 6).

Overall, FIG. 1 provides a perspective view of the cobot 100 in operation. In use, the cobot may be stationed near an operating table (outside the sterile field) and can reposition itself as needed during a procedure. The humanoid cobot embodiment (with arms 106) may, for instance, stand adjacent to the anesthesia provider, with one robotic arm ready to retrieve a medication from the storage module 120 and hand it to the provider. The alternate wheeled-only cobot 100 may similarly move to the provider’s vicinity and present an open compartment 122 containing the requested medication for the provider to take. Both versions include safety features (like rounded edges, protective coverings, and compliance with collision force limits) to allow close proximity to humans in a collaborative manner. The components shown in FIG. 1, including the mobile base 102, robotic limbs 106, storage module 120, HMI 130 (touchscreen 132 and authentication devices), and sensors 140, all work together under the control of onboard electronics to achieve secure, context-aware medication dispensing in the OR, as further detailed in subsequent figures.

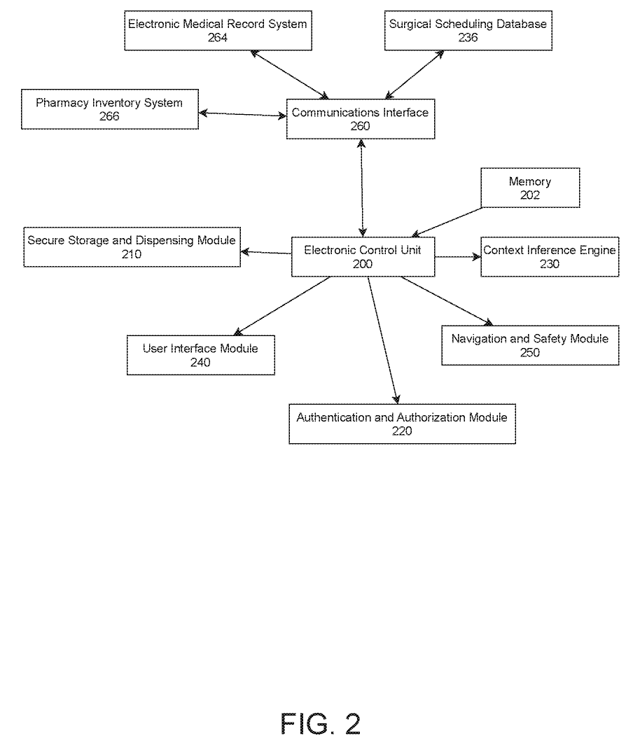

Referring now to FIG. 2, a block diagram of the cobot’s hardware and software architecture is presented. The central controller 200 (for example, an embedded computer or processor with associated memory 202) orchestrates all operations of the cobot. The controller 200 is programmed with the various functional modules needed for medication control and dispensation, and it interfaces with all hardware components. Key subsystems depicted in FIG. 2 include:

Secure Storage and Dispensing Module 210: This corresponds to the physical medication storage unit 120 of FIG. 1. It includes electronically controlled locks or latches for each compartment 212, as well as dispense actuators 214 (e.g. motorized drawer mechanisms or an elevator system that can deliver a selected dose to an output slot). The module 210 is connected to inventory sensors 216 that monitor remaining stock and detect when a medication has been removed. The controller 200 can activate a specific actuator 214 to unlock/open a compartment when a dispense command is authorized, while keeping all other compartments secured. Feedback signals from the storage module (such as a limit switch indicating a drawer is open, or a sensor confirming item retrieval) are sent to the controller to confirm execution of dispensing.

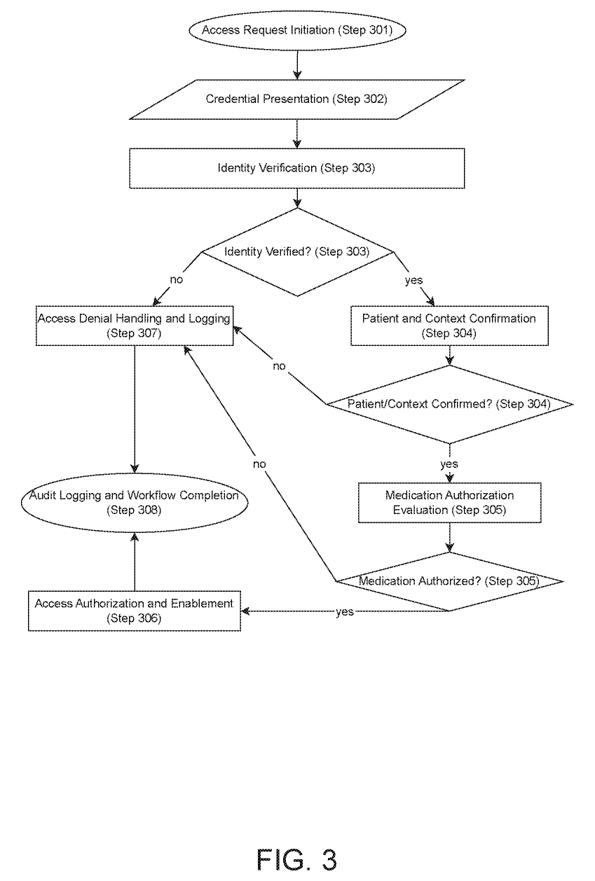

Authentication and Authorization Module 220: This block encompasses the multi-factor authentication interface and logic. Hardware elements such as the badge reader 136, biometric scanner 134, and PIN input device are represented, feeding into an identity verification unit 222. The controller’s software includes an authentication workflow (as detailed in FIG. 3) that requires one or more factors to match authorized personnel credentials stored in a secure staff database 224 (which may be stored locally in memory 202 and/or accessed from a hospital server). Only upon successful authentication does the authorization module 220 enable medication access. The same module (or a closely linked control subroutine) also handles dispensation authorization by cross-referencing patient data and medication orders via the EMR integration (for example, confirming that the user’s requested medication is on the patient’s prescription list and appropriate for the current surgical context, as will be described). In essence, module 220 enforces that (1) the person accessing the meds is authorized, and (2) the medication being dispensed is authorized for the patient and timing.

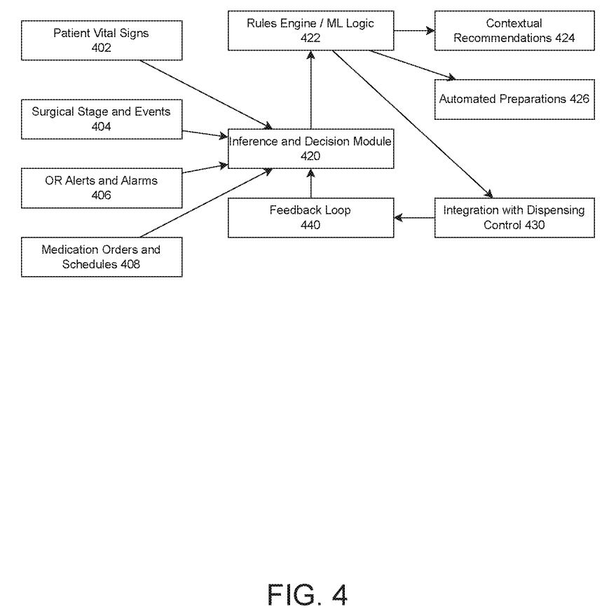

Context Inference Engine 230: This is the software module that processes contextual inputs to guide dispensing logic (corresponding generally to the system of FIG. 4). It receives data from various sources, such as patient vital sign monitors 232, operating room equipment and sensors 234 (including signals like “surgery start” or “bypass machine alert”), and the surgical schedule database 236. The inference engine 230 uses this data to assess the current stage of the procedure and patient condition. It may run rule-based algorithms or machine-learning models to predict medication needs – for example, recognizing from vital trends that blood pressure is dropping rapidly and suggesting a vasopressor medication. The output of this engine can trigger alerts or recommendations on the HMI 130 and can even pre-arm the dispensing module for rapid access to likely-needed drugs (pending user confirmation).

User Interface Module 240: This encompasses the touchscreen display 132, the voice recognition subsystem 242, and output devices like speakers or indicator lights. It manages the interactive dialogues with the clinician. For instance, it generates the prompts for login (FIG. 3), displays lists of available medications or patient information, and handles voice command processing. The UI module 240 is designed for usability in the OR: large, clear on-screen buttons, minimal required steps for critical tasks, and an option for hands-free operation via voice commands. The module also includes a feedback component that provides confirmation messages (visual and/or auditory) when actions are taken (such as “Morphine 2 mg selected – awaiting confirmation” or “Dispense complete – please remove medication”). FIG. 2 shows this module interfacing with both input devices (touch, voice) and output devices, as well as linking to the authentication module 220 and context engine 230 to present relevant information to the user.

Navigation and Safety Module 250: This block represents the cobot’s locomotion control and safety systems (detailed in FIG. 6). It includes the drive system 252 (motors, wheel controllers) and mobility sensors 254 (e.g. cameras, LiDAR, ultrasonic sensors for obstacle detection). The module 250 runs path planning and obstacle avoidance algorithms, enabling the cobot to move autonomously in the OR without collisions. It also enforces sterile field boundaries by utilizing inputs from vision sensors and predefined maps (or coordinates) of sterile zones 256. For example, the controller 200 will create exclusion zones in its navigation map corresponding to the sterile field and will plan routes that circumvent those zones. Real-time sensor data is analyzed for any potential breach – if, for instance, a robotic arm or the base gets too close to a sterile-draped area, the safety module can halt movement and alert the user. This module also covers emergency stop logic and compliance with safety standards (the cobot can automatically stop if unexpected resistance or contact is detected, preventing any unsafe force against people or equipment).

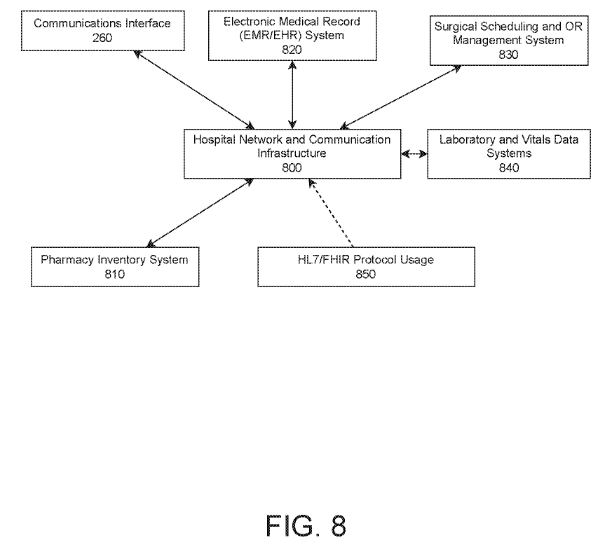

Communications Interface 260: The diagram in FIG. 2 shows a wireless communication module 260 that links the cobot to external hospital systems and networks (see FIG. 8). This typically includes a secure Wi-Fi or dedicated wireless link 262 for data exchange with servers such as the hospital’s electronic medical record (EMR) system 264, pharmacy inventory system 266, and others (e.g., scheduling system 236 as mentioned, or an audit log server 268). The communications interface handles standard protocols for healthcare interoperability – for example, it may transmit and receive HL7 or FHIR formatted messages to query patient data and update medication administration records. It also ensures that all data transmissions (especially patient information and medication logs) are encrypted and comply with hospital IT security policies. The communications module allows the cobot to function as part of the larger healthcare information ecosystem, updating databases in real time and retrieving the latest orders or alerts.

Each of these subsystems in FIG. 2 is operatively connected through the central controller 200. In operation, when a clinician interacts with the cobot, the controller coordinates the authentication sequence (module 220), then consults context and authorization rules (230 and 220) before actuating the dispensing hardware (210) and logging events via the communications link (260). Simultaneously, if the cobot needs to move (for example, to approach the clinician for hand-off or to move out of the way), the navigation module 250 takes command under the controller’s oversight. FIG. 2 thus provides a high-level view of how hardware and software components are integrated to deliver the functionality described in this disclosure.

Medication access in the perioperative cobot system is tightly controlled through a multi-factor clinician authentication process, illustrated in the flowchart of FIG. 3. This process ensures that only authorized personnel can retrieve medications and that medications dispensed by the system are verified against patient orders and procedural context prior to release. FIG. 3 depicts a sequence of interactions and validation checks beginning when a clinician initiates a request for medication access from the cobot and proceeding through authentication, authorization, and access control steps, as described below.

Access Request Initiation (Step 301). The workflow begins when a clinician, such as an anesthesiologist or circulating nurse, signals an intent to access medication from the cobot. This request may be initiated by interacting with the touchscreen display 132 (for example, selecting a “Wake” or “Unlock” control) or by issuing a voice command (e.g., “Robot, I need medication”). In response, the cobot transitions into an authentication mode and prompts the clinician to identify themselves, which may be visually displayed on the touchscreen and optionally accompanied by an audible voice prompt.

Credential Presentation (Step 302). After access request initiation, the clinician provides the required authentication credentials. In a preferred configuration, at least two distinct authentication factors are required, and in higher-security configurations all three available modes may be employed. By way of example, the clinician may scan an identification badge using the badge reader 136, provide biometric input via the fingerprint scanner 134, and/or enter a personal identification number (PIN) using the touchscreen 132. The cobot supports multiple combinations of authentication factors, such as badge plus PIN, biometric plus PIN, or badge plus biometric, in accordance with hospital policy. Each credential input is captured by the system and forwarded to the authentication module 220 for evaluation.

Identity Verification (Step 303). The electronic control unit 200 verifies each received authentication factor against stored credential data or via a secure network query. Badge identifiers are compared against authorized clinician records, biometric inputs are matched to stored biometric templates, and PINs or passwords are validated against encrypted credential hashes. Secure processing techniques may be employed, including local biometric verification, to protect sensitive data. If any required authentication factor fails verification, access is denied and the clinician may be prompted to retry or seek administrative assistance. Upon successful verification of all required factors, the clinician’s identity and a timestamp are recorded in an audit log.

Patient and Context Confirmation (Step 304). Following successful clinician identity verification, the cobot confirms patient identity and surgical context prior to permitting medication selection. In many implementations, the cobot is already associated with a specific patient or procedure based on initialization data received from a surgical scheduling system. At this step, the system cross-checks patient identity and current surgical stage by querying the electronic medical record (EMR) system or receiving context updates from connected operating room systems. The clinician may be prompted to confirm patient identity via the touchscreen if multiple patients are possible. Once confirmed, the system retrieves active medication orders for the identified patient using standard interoperability protocols such as HL7 or FHIR.

Medication Authorization Evaluation (Step 305). After patient context is confirmed, the cobot evaluates whether a requested medication is authorized for dispensation at the current stage of the procedure. The clinician may explicitly select a medication via the user interface or issue a voice command identifying the medication. In other scenarios, the context inference engine 230 may generate a medication recommendation, which the clinician can accept or decline. Prior to unlocking any storage compartment, the system verifies that the selected medication and dosage are consistent with active medication orders, standing protocols, and predefined clinical rules. If authorization cannot be confirmed, the system issues an alert or requires an override confirmation, thereby preventing unauthorized or inappropriate medication dispensation.

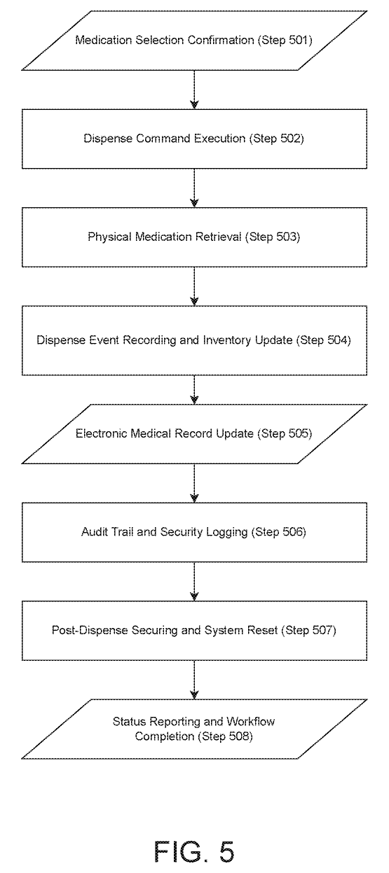

Access Authorization and Enablement (Step 306). If both clinician authentication and medication authorization checks are successfully completed, the electronic control unit grants access to the medication dispensing function. The user interface reflects this authorization state, for example by enabling medication selection controls or confirming a previously selected medication. The clinician is then permitted to proceed with medication retrieval, with dispensing operations performed as described in connection with FIG. 5. Authentication status may remain active for a configurable duration, although the system may require re-authentication for subsequent dispensation events or after a defined period of inactivity.

Access Denial Handling and Logging (Step 307). If any authentication, patient confirmation, or authorization step fails, the system denies access to the medication storage module and records the denial event in the audit log. The clinician may be notified of the reason for denial via the user interface, such as invalid credentials or lack of medication authorization for the patient or procedure stage.

Audit Logging and Workflow Completion (Step 308). Throughout the FIG. 3 workflow, the system records relevant data including clinician identity, patient identity, authentication outcomes, authorization results, and timestamps as part of a secure audit trail. Upon successful completion of the authentication and authorization sequence, control transitions to subsequent context-aware decision support and medication dispensing operations described with reference to FIGS. 4 and 5.

FIG. 4 depicts a schematic diagram of the context-aware dispensing logic employed by the cobot system. In essence, this part of the system uses real-time data and situational awareness to intelligently support medication dispensing during surgery. The goal is to ensure that medications are not only secure and authorized (as handled by FIG. 3) but also timely and appropriate given the dynamic conditions of the surgery and patient status. The context-aware logic (implemented by context inference engine 230 in the controller) integrates multiple streams of information to anticipate needs and optimize workflow, as outlined below with reference to FIG. 4:

Input Streams: The left side of FIG. 4 represents various input data sources feeding into the cobot’s inference engine. Key inputs include:

Patient Vital Signs 402: The cobot receives continuous or periodic data from patient monitoring devices (such as heart rate, blood pressure, oxygen saturation, respiratory rate, etc.). This can be achieved through a direct interface with the anesthesia monitor or via the hospital network (for example, using a standard like IEEE 11073 or a vendor-specific API to obtain vitals). Abnormal trends or threshold crossings in these vitals are flagged by the system. For instance, a sudden drop in blood pressure might signal that a vasopressor drug will be needed imminently, or a high heart rate with fever might indicate the need for an antipyretic or cooling measures.

Surgical Stage and Events 404: Information about the progress of the procedure is provided through the surgical scheduling system or manual input from OR staff. The system knows the scheduled procedure and can infer stages such as “induction of anesthesia,” “incision start,” “critical phase (e.g., cardiopulmonary bypass on/off),” and “closing.” This could be as simple as time-based stages from the schedule (e.g., prophylactic antibiotics should be given within one hour of incision) or as sophisticated as real-time recognition of surgical phases via sensors/cameras in the OR (in some advanced systems). The cobot may also accept direct signals – for example, an integration such that when the first incision is made, an event is sent to the cobot. These cues allow the system to align medication dispensing with standard perioperative protocols (e.g., indicating when to provide additional anesthesia or antibiotics).

OR Alerts and Alarms 406: The cobot taps into the OR’s clinical alert streams. This includes alarms from devices (like ventilator alarms, anesthesia machine alarms for low agent or high pressure, etc.), as well as hospital-wide alerts (such as a code blue or anaphylaxis alert in that OR). If a critical alarm sounds, the cobot’s logic interprets it in context – for example, an “arterial oxygen desaturation” alarm might prompt the system to prepare a syringe of a bronchodilator or emergency medication like epinephrine, anticipating an intervention for a possible respiratory issue.

Medication Orders and Schedules 408: Beyond just checking orders for authorization, the system continually monitors the patient’s medication schedule. For instance, if the patient is due for a next dose of an ongoing medication (like a redosing of an antibiotic after a certain interval during a long surgery), the system will recognize the timing. If the surgeon has requested a medication via the EMR (or if the anesthesiologist entered an order on their workstation), the cobot can receive that order in real time. This ensures the robot is aware of any new or changed medication requirements without the clinician having to manually input them on the cobot.

Inference and Decision Module 420: At the heart of FIG. 4, the context inference module (420) processes the above inputs using a set of rules or AI models (represented by logic blocks 422). For example, the system can have a rules engine that says: “If systolic BP < X and dropping, and if an appropriate vasopressor is ordered or available, then alert the user and suggest preparing that medication.” Another rule could be: “If current stage is ‘closing’ and if no hemostatic agent has been given, consider suggesting one if bleeding is above threshold.” These rules can be defined based on clinical guidelines or learned from past data. In some embodiments, the system employs machine learning to refine its suggestions, for example, it might learn typical medication usage patterns for certain surgeries and pre-emptively have those meds ready. The output of this inference module is contextual recommendations 424 and automated preparations 426.

Recommendations 424: These are non-binding prompts to the clinical user. The cobot might display a message like “Recommend preparing epinephrine – patient heart rate unstable” or verbally announce “Consider antibiotic re-dose now.” The recommendation engine prioritizes safety (it might recommend but not automatically act unless confirmed by a human).

Automated Preparations 426: In some scenarios, the cobot can take preparatory actions without immediate human command, while still keeping the final control with the clinician. For instance, upon detecting that incision is made (stage change to intra-operative), the cobot could automatically unlock the compartment containing the prophylactic antibiotic or move it to a ready position (but perhaps not open it fully until a clinician confirms). Another example: if a life-threatening arrhythmia is detected via patient monitors, the cobot might go as far as unlocking and illuminating the compartment with the emergency drug (e.g., anti-arrhythmic or epinephrine) to save precious seconds, while issuing a voice alert to the team. The system can be configured for how aggressive these automated steps are, ensuring that the final dispensation still requires a human action (to remove the drug or confirm the dispense) to maintain proper control.

Integration with Dispensing Control 430: FIG. 4 shows the outputs of the inference engine feeding into the dispensing control logic. This means the recommendations or preparatory actions are tied into the FIG. 5 workflow. For example, if the context engine has pre-validated a medication need and readied the compartment, the dispensing workflow can proceed faster once the clinician gives the go-ahead (some checks may be skipped because they were pre-done). The controller 200 coordinates so that any context-based decision still goes through the authorization checks (for safety) before the compartment opens.

Feedback Loop 440: The context-aware system also refines itself through feedback. After a medication is dispensed, the system notes whether it was prompted by a recommendation or by direct user request. It can learn from cases where it made a suggestion and the user accepted or ignored it, adjusting future behavior accordingly. Additionally, the system monitors outcomes: if a suggestion was followed by a positive patient response (e.g., blood pressure stabilized after suggested drug given), that rule may be reinforced. FIG. 4’s diagram might illustrate this feedback loop where data from the outcome (patient vitals post-medication and user actions) feeds back into the inference module 420 for continuous improvement.

By leveraging these context inputs and intelligent rules, the cobot adds a layer of situational awareness beyond a simple medication cabinet. It essentially serves as a smart assistant: not only does it store and dispense medications, but it also “keeps an eye” on patient condition and surgical progress. This context-aware logic helps in anticipating clinicians’ needs – for example, having the next syringe ready so the provider doesn’t have to break focus to search for it, thereby improving response time during critical moments. It’s important to note that while the cobot can make recommendations or preparations, the surgical team remains in control at all times; the system is an aid, not an autonomous decision-maker on medication administration. In summary, FIG. 4 shows how environmental and patient data are synthesized in real time to enhance decision support for medication dispensing, aligning the cobot’s actions with the current perioperative context.

Once a clinician is authenticated in accordance with FIG. 3 and a medication request is confirmed, potentially with assistance from the context-aware logic of FIG. 4, the cobot proceeds with the medication dispensation process and associated event logging. FIG. 5 presents this process in a spreadsheet-style sequence illustrating the ordered operational steps from medication selection through completion of dispensing, documentation, and post-dispense handling. The steps shown in FIG. 5 encompass both the mechanical dispensing actions performed by the cobot and the software-driven logging, inventory, and communication operations, as described below.

Medication Selection Confirmation (Step 501). The clinician confirms the desired medication and dosage via the human-machine interface (HMI) 130. Selection may be performed by touching a medication entry on the touchscreen or by issuing a voice command. If the context inference engine previously generated a medication recommendation, the interface may present a single-action confirmation control (for example, “Dispense 1 ampule of epinephrine now?”). Upon confirmation, the system identifies the specific medication and dosage to be dispensed. As a final safeguard, the electronic control unit may perform an additional authorization check to confirm that the selected medication and dosage remain authorized for the patient at the current procedural stage.

Dispense Command Execution (Step 502). After medication selection confirmation, the electronic control unit 200 issues a dispense command to the secure storage and dispensing module 210. The control unit actuates the dispensing mechanism associated with the compartment 122 storing the selected medication, unlocking and opening only the specific compartment containing the authorized medication. Visual indicators may be activated on the HMI to guide the clinician to the opened compartment. All other medication compartments remain locked to prevent unauthorized access or selection errors. Where applicable, the dispensing mechanism measures and releases a predetermined dosage of the medication.

Physical Medication Retrieval (Step 503). With the designated compartment open, the clinician retrieves the medication dose. In embodiments including an articulated robotic arm 106, the cobot may grasp the medication container from the storage module 120 and present it to the clinician at a convenient handoff position. The robotic arm operates with controlled motion and releases the medication only upon detecting clinician grasp. In wheeled-only embodiments, the clinician manually removes the medication from the open compartment. One or more sensors detect removal of the medication. If the dispense action is canceled or the medication is returned, the system re-secures the compartment and records the event accordingly.

Dispense Event Recording and Inventory Update (Step 504). Immediately following medication retrieval, the electronic control unit records details of the dispensation event. Logged data may include patient identity, medication name, dosage, time and date of dispensation, clinician identity, and contextual metadata associated with the procedure. The system updates inventory counts for the dispensed medication and determines remaining stock levels within the storage module. If inventory levels fall below a predefined threshold, the system may generate a restocking notification or alert.

Electronic Medical Record Update (Step 505). After recording the dispense event locally, the cobot transmits dispensation data to one or more hospital information systems, including the electronic medical record (EMR) or electronic medication administration record (eMAR). Communication may occur using standardized healthcare interoperability protocols such as HL7 or FHIR. Depending on configuration, the transmission may document medication removal, medication administration, or both, thereby synchronizing patient records, pharmacy systems, and billing systems in near real time.

Audit Trail and Security Logging (Step 506). In parallel with EMR updates, the system records the dispensation event in a secure audit log stored in memory 202 and optionally synchronized with a remote server. The audit trail includes clinician identity, timestamps, medication details, and system status indicators. Any anomalies detected during the dispense process, such as prolonged compartment access or aborted dispense attempts, are also logged. For controlled substances, the audit log supports regulatory compliance, inventory reconciliation, and periodic audits.

Post-Dispense Securing and System Reset (Step 507). Following completion of logging and data synchronization, the cobot automatically closes and locks the accessed compartment. The HMI may display a confirmation message indicating successful completion of the dispense event. The system then transitions to a standby state, maintaining an authenticated session for a configurable duration or requiring re-authentication based on hospital policy. If the procedure is concluding, the clinician may log out or the system may automatically time out.

Status Reporting and Workflow Completion (Step 508). Upon completion of the dispensing sequence, the cobot may transmit summary status information to pharmacy or inventory management systems, including a record of medications dispensed during the procedure and any restocking or maintenance requirements. FIG. 5 thus represents a complete, linear workflow capturing both the physical dispensing sequence and the associated information management operations, ensuring accountability, traceability, and operational efficiency in perioperative medication management.

FIG. 6 illustrates the cobot’s approach to navigating within the operating room and maintaining compliance with sterile field protocols. The operating room is a crowded and dynamic environment, with surgeons, nurses, anesthesiologists, and equipment all present. The cobot must move safely without impeding the surgical workflow or violating the sterile field (the area immediately around the patient and surgical site that must remain free of contamination). The diagram in FIG. 6 can be understood as a top-down layout of an OR or a schematic of the cobot’s positioning system, highlighting key safety zones and path planning logic. The following aspects are addressed in FIG. 6:

Defined Zones and Sterile Field Boundary: In the figure, the sterile field 600 is indicated (for example, a circular or rectangular area around the operating table). The cobot is programmed with knowledge of this zone – it may be configured before the procedure with the location of the sterile field, or it may dynamically detect it via sensors. For instance, the robot’s vision system can recognize the blue sterile drapes on the patient and instrument tables, or it could use fiducial markers that are placed at sterile boundaries. Another approach is that the OR has a mapped layout where certain coordinates are designated as sterile-only. However it’s determined, the cobot establishes a virtual boundary 602 around this area which it will not cross. FIG. 6 might depict the cobot 100 with a planned path 610 that carefully skirts the sterile boundary line 602. If a part of the cobot (such as an extended arm) needs to reach toward the sterile area (for example, handing something to a scrub nurse), the system ensures that only the sterile item crosses the boundary (perhaps by the nurse grabbing it while the robot’s arm stays at the edge). The cobot itself does not intrude into the sterile field – its wheels stay outside, and it avoids contact with sterile surfaces. If, due to any error or unforeseen movement, the cobot gets too close, the system either stops or alerts staff to intervene, thereby preventing contamination.

Dynamic Obstacle Avoidance: FIG. 6 shows various obstacles such as OR staff 620 (circulating nurse, surgeons) and equipment carts 622 in the room. The cobot uses its sensor suite 254 to detect these obstacles in real time. The navigation module 250 employs algorithms (e.g., LiDAR-based mapping and sonar for proximity) to navigate around people and objects. If a person suddenly steps into the cobot’s path, the robot will either slow down, stop, or re-route as appropriate. The path planning logic might use waypoints around the OR – for example, it knows a typical station for the anesthesiologist (near the head of the patient) and can plan a route from its charging corner to that station while avoiding known no-go zones. In FIG. 6, the cobot’s path 610 curves around any obstacles rather than straight lines through them. The system can utilize classic robotic navigation techniques (such as A* or Dijkstra for path planning on a grid of the room, combined with real-time obstacle avoidance using methods like the Dynamic Window Approach or others). The result is that the cobot can autonomously drive itself to desired locations in the OR, for example: approaching the anesthesiologist when a medication is requested, or moving aside to a wall if not in use.

Positioning for Handoffs: FIG. 6 illustrates specific positions such as a “handoff position” 630 at the edge of the sterile field near the anesthesia workspace. The cobot is aware of these designated spots, which are optimal for delivering medications (close enough for the clinician to reach, but outside critical zones). In the humanoid arm embodiment, the cobot might stop a short distance away and then use its arm to bridge the gap, as shown by an extended arm icon reaching toward a person. In the wheeled-only embodiment, the cobot might come slightly closer so that its compartment is within reach of the nurse or anesthesiologist. In either case, the cobot’s precise positioning system (potentially using visual landmarks or distance sensors to, say, align itself with a reference point on the floor or ceiling) ensures it doesn’t bump into the OR table or other devices.

Sterile Field Monitoring: In some embodiments, the cobot is not only avoiding the sterile field, but actively monitoring it for compliance (though primary responsibility lies with humans, the robot can add an extra set of “eyes”). For instance, using its camera, it might detect if a non-sterile object breaches the sterile field and issue an alert, similar to systems described by UCF researchers where sensors track events that violate sterile boundaries. This is an auxiliary feature – the cobot could notify OR staff if it “sees” something like an unsterile sleeve or instrument tip touching a sterile area. That extends the safety functionality beyond the cobot’s own movement to the general OR practice. This concept is depicted in FIG. 6 by showing the cobot’s vision cone covering the sterile field area, with an indicator that it can detect unauthorized intrusions.

Collision Avoidance and Safety Stops: The cobot’s navigation system adheres to the rigorous safety requirements of collaborative robots. FIG. 6 may include a flow or note such as “Obstacle detected –> Stop or reroute.” For example, if a person suddenly backs into the cobot or a piece of equipment is wheeled into its way, the cobot has built-in collision detection. This could be as simple as bump sensors around its base (that immediately cut motion if contact is made) and as advanced as predictive stopping using vision (recognizing a moving person and preemptively pausing). The cobot travels at a moderate speed cap in the OR (slow enough to stop quickly if needed). The safety module 250 ensures that the force of any accidental contact is below harmful thresholds (the robot might be lightweight or have force sensors to detect pressure). FIG. 6 might symbolically show a protective bubble around the cobot indicating its personal space for safe operation.

Navigation in the Presence of Sterile and Non-sterile Personnel: In an OR, some staff (surgeons, scrub nurses) are sterile and confined to the sterile field, while others (anesthesiologist, circulator) are not sterile and move more freely. The cobot is programmed to primarily interact with the non-sterile personnel (since they can touch the robot without issue). For instance, it will approach the anesthesiologist for medication handoff rather than a scrub nurse. If a scrub nurse needs a medication (say a sterile saline syringe for field irrigation), typically they would ask the circulator to get it from the robot. The robot thus positions itself to facilitate that exchange efficiently. FIG. 6 might label zones like “Circulator zone” vs “Surgeon zone,” with the cobot staying in the circulator’s domain.

Alternate Embodiment Differences: The wheeled-only version of the cobot 100 follows the same navigation principles as the humanoid version. However, because it cannot extend an arm, it might need to come slightly closer to the user for item retrieval. The system takes that into account by perhaps allowing the wheeled unit to approach the anesthesia table more closely (still without touching anything sterile). The difference could be a matter of inches but is managed carefully. In either case, neither version will ever roll over hoses or wires on the floor if it can help it – the path planning can factor in known floor hazards (like ECG cables or anesthetic gas tubing often found around the patient). If the cobot detects an obstacle on the floor that wasn’t there before (like a footstool or dropped tool), it will adjust course.

FIG. 6 thus demonstrates that the cobot actively maintains spatial awareness and safety as it navigates. Through a combination of pre-configured constraints (maps of OR layout and sterile zones) and real-time sensor feedback, the robot ensures it integrates seamlessly into the surgical environment without causing contamination or collisions. The ability to autonomously navigate to the right place at the right time, for example, coming when called for a medication and then retreating out of the way, adds to workflow efficiency. At the same time, features like sterile field monitoring and obstacle avoidance provide an extra layer of safety and reliability, which is crucial in the high-stakes setting of an operating room.

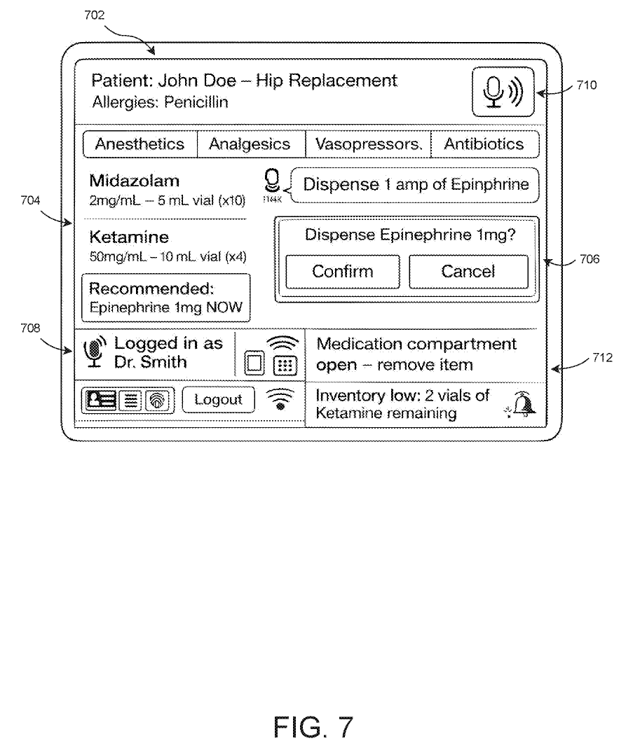

FIG. 7 provides a detailed view of the human-machine interface (HMI) 130 of the cobot, illustrating the layout of the user interface and the various modalities through which clinicians can interact with the system. The HMI is designed to be intuitive and to accommodate the fast-paced, sometimes hands-busy context of surgery. In FIG. 7, one can see an example touchscreen display 132 with several functional regions, as well as indicators for voice control and system status. Key elements of the interface include:

Display of Patient and Case Information: A header area 702 on the touchscreen shows the current patient’s name or ID, along with key context info such as the procedure name and perhaps allergies or other alerts (e.g., “Patient: John Doe – Hip Replacement; Allergies: Penicillin”). This confirms to the user that the cobot is correctly linked to the right patient’s records, and provides at-a-glance information that might be relevant when choosing medications (for instance, highlighting an allergy is crucial to avoid dispensing that antibiotic).

Medication Selection Menu: The central portion of the UI 704 lists the medications that are available/authorized for this case. Medications might be grouped by categories (tabs or sections for “Anesthetics,” “Analgesics,” “Vasopressors,” “Antibiotics,” etc.) for quick access. Each listed item could display the drug name, concentration, and forms available. For example, an entry might read “Midazolam 2mg/mL – 5 mL vial (x10 available)”. If the context-aware system has recommendations (from FIG. 4), those might appear at the top of the list or highlighted (e.g., “Recommended: Epinephrine 1mg NOW” might be a flashing or colored entry if the patient is crashing). The user can touch a medication entry to select it; if a dose choice is needed, a sub-menu or prompt appears. In many cases, each item corresponds to a single standard dose so it might directly queue that for dispense.

Controls for Dispensing and Confirmation: Once a medication is selected, the interface might show a confirmation dialog 706 (especially if using touch input). This dialog could say “Dispense Morphine 2mg?” with options to confirm or cancel. This helps prevent accidental activations. If voice command is used, the system similarly will ask for confirmation either verbally or by showing the intended action on screen with a prompt to confirm (the clinician can say “Confirm” or press a button). There may also be an option to adjust the quantity if multiple doses are needed (e.g., entering a number or using +/- buttons if the clinician wants 2 units of something).

Authentication and User Controls: On FIG. 7, a section 708 might be dedicated to user authentication status and actions. For instance, if not logged in, it would show “Please scan your badge or fingerprint to begin” with corresponding icons. If already logged in, it could display the name of the authenticated user (e.g., “Logged in as Dr. Smith”) and a logout button. The PIN pad for password entry could pop up in this area during the login process (as described in FIG. 3 workflow). The interface might also allow the user to re-authenticate quickly if a high-security med is selected that requires additional confirmation (e.g., a controlled substance might prompt for a secondary fingerprint scan just for that transaction as an extra measure).

Voice Control Indicator: A microphone icon 710 or similar indicator on the screen shows the status of voice control. When the icon is active (lit up), it means the system is listening for voice commands. The user might say a wake word or press the icon to initiate a voice interaction. The cobot’s voice recognition 242 is configured to understand medication names and simple commands (“dispense,” “confirm,” “cancel”). For example, a clinician could say, “Robot, dispense 1 amp of epinephrine.” The system would then repeat back, “Dispense epinephrine 1 milligram, correct?” awaiting a confirmation. FIG. 7 depicts text feedback of the voice command as well (to ensure the system heard correctly). Voice control is particularly useful when the clinician’s hands are occupied (such as during a critical event where manual interaction with the screen would be too slow or disruptive). The system uses a medical vocabulary tuned to drug names to reduce errors in recognition. If it’s unsure of a command, it will ask for clarification (e.g., “Did you mean ephedrine or epinephrine?” because such names can sound similar). The microphone icon might blink while listening and a separate speaker icon could indicate when it’s speaking.

System Status and Alerts: FIG. 7 also shows an area 712 for system messages and alerts. This is where the cobot communicates information like “Medication compartment open – remove item” or “Inventory low: 2 vials of Ketamine remaining.” It also displays safety alerts such as “Obstacle detected – movement paused” or “Sterile field proximity alert” if those events occur (though critical safety alerts might also be signaled by an audible alarm or spoken warning). In addition, after a medication is dispensed, a message might confirm “Dispensed [Drug] at [Time] – Logged”. These messages keep the user informed of the cobot’s status and any actions they might need to take. The status area might also have icons for battery level (since the cobot is mobile and battery-powered) and network connectivity, so staff can ensure the robot is charged and connected.

User Input/Output Hardware: FIG. 7 is focused on the touchscreen, but we note the presence of physical hardware that complements it. For example, the fingerprint scanner 134 might be located just below the screen (with an icon on screen guiding the user “Place finger here”). The badge reader 136 might be on the side of the screen or base, but an on-screen prompt will say “Tap badge now.” There could also be physical emergency stop or pause buttons on the cobot, which are not part of the touchscreen UI but are visible on the robot – pressing those would immediately halt movement or dispensing in case of any hazard or to comply with a human command. Those are typically marked in red and shown in diagrams like FIG. 1, but not on FIG. 7 which is specifically the screen interface.

Ease of Use Features: The interface uses large fonts and high contrast colors for readability in the OR (where lighting may be low during some procedures). It has a dark mode to avoid glare. The touch targets are large enough to be used even with gloves (non-sterile gloves, or perhaps through a sterile cover if needed). Voice feedback is delivered at a volume that the user can hear over background noise but not so loud as to be disruptive. For instance, it may have a “quiet mode” during critical surgical moments, only displaying silent notifications unless something urgent happens.

Overall, FIG. 7 demonstrates that the cobot’s user interface is thoughtfully designed for the surgical setting, providing multiple ways for clinicians to interact – via touch, voice, and secure logins – with minimal distraction. It integrates the necessary functions: authentication, medication selection, confirmation, and feedback. By making the interface user-friendly and context-aware (e.g., showing relevant info and recommendations), the system ensures that clinicians can quickly and safely get the medications they need without navigating a complicated system. This is vital in the OR, where every second counts and ease of use can directly impact patient outcomes.

FIG. 8 is a diagram illustrating how the cobot system interfaces with various hospital information systems through wireless networking and standard data protocols. This integration enables the cobot to function not as an isolated unit, but as a connected part of the hospital’s clinical and administrative workflow. By syncing data with existing systems (such as pharmacy inventory databases, electronic health records, and surgical scheduling systems), the cobot ensures consistency of information and facilitates real-time updates. The components of the integration shown in FIG. 8 include:

Hospital Network and Communication Infrastructure 800: At the center of FIG. 8, there is a representation of the hospital’s secure network (for example, a cloud or server icon). The cobot’s communication module 260 connects wirelessly to this network using Wi-Fi or a dedicated wireless protocol. All data transmitted is encrypted (using protocols like WPA2 Enterprise Wi-Fi security and additional encryption layers for sensitive data) to comply with HIPAA and hospital cybersecurity standards. The diagram shows the bidirectional data flow between the cobot and the various systems over this network.

Pharmacy Inventory System 810: One important integration is with the central pharmacy or inventory management system. The cobot sends inventory updates to this system whenever medications are dispensed (as described in FIG. 5’s logging step). For example, if one vial of drug X was dispensed, the cobot’s message might decrement the count of drug X in the OR’s inventory record. Conversely, the pharmacy system can update the cobot – for instance, when a pharmacy technician refills the cobot’s storage, the updated inventory levels can be pushed to the cobot’s database. FIG. 8 might show an arrow from the cobot to the pharmacy DB labeled “Usage data” and an arrow back labeled “Restock info”. By maintaining synchronization, the hospital avoids stock-outs or overstock in the cobot: the pharmacy knows exactly what and when to replenish. Integration examples include HL7 messaging (e.g., an HL7 ORM or DFT message for a dispense event) or FHIR resources (InventoryItem or MedicationDispense entries) being updated. The system may also integrate with automated pharmacy systems like Omnicell or Pyxis servers, reporting itself similar to an ADC unit on the network.

Electronic Medical Record (EMR/EHR) System 820: The cobot interfaces with the EMR to exchange clinical information. Prior to dispensing, it retrieves patient data and orders (as discussed, possibly using FHIR API calls or HL7 messages to get the medication orders for the patient). After dispensing, it can send documentation (like a message that a dose was dispensed at a certain time). Many EMR systems support receiving such messages to populate the patient’s medication administration record automatically. FIG. 8 shows a data flow where the cobot queries the EMR for “Medication Orders / Allergies / Patient ID” on one side, and on the other side sends “Dispense log / Administration record”. For example, upon dispensing, the cobot might create a FHIR MedicationAdministration resource or send an HL7 RAS message (Pharmacy Administration Message) to record that the medication was given. In this way, the medication event is captured in the patient’s official record without manual transcription. Additionally, integration with the EMR means the cobot can receive real-time updates such as a cancellation of an order or a new stat order placed – ensuring it acts on the most current information.

Surgical Scheduling and OR Management System 830: Before and during a case, the cobot can get information from the scheduling system about the procedure. FIG. 8 indicates an interface to a scheduling database or OR management platform which holds the surgery start time, type of procedure, assigned staff, etc. This info is used by the cobot to set up its context (like selecting the correct patient and loading relevant protocols). It might receive a message when the case is officially started or when the patient is moved to the OR, which can trigger the cobot to activate and move to the OR if it’s stationed outside. Similarly, if a case is delayed or moved, the schedule integration prevents the cobot from showing up at the wrong time or place. This system may communicate via HL7 SIU messages (Schedule Information Unsolicited) or through an API that the cobot polls for updates.

Laboratory and Vitals Data Systems 840: Some hospitals have centralized vitals streaming or lab results systems. The cobot can optionally integrate here to enrich its context: for example, pulling recent lab results (like arterial blood gas or hemoglobin levels) if that might influence medication needs (e.g., if potassium is low, be ready for a KCl infusion). Or integrating with a vitals aggregator that broadcasts patient monitor data – this could be an alternative path to get vitals if direct device integration isn’t possible. FIG. 8 might not explicitly show this, but it can be considered part of the overall network the cobot can access if needed.

HL7/FHIR Protocol Usage 850: FIG. 8 lists the standards by which these integrations occur. The cobot’s software can parse HL7 v2 messages which are common in hospital systems (for example, an HL7 ADT message to know the patient’s admission and OR transfer, an HL7 ORM for new medication orders, and HL7 RAS/RAD for medication administrations). It can also use FHIR, which is a modern RESTful API standard, to GET or POST resources (like MedicationRequest, MedicationAdministration, InventoryItem, etc.). By supporting these standards, the cobot can communicate with a wide range of systems out-of-the-box, since many vendors and systems use HL7 and FHIR for interoperability. Implementing these standards facilitates a seamless exchange of health information between the cobot and external systems, which improves overall workflow coordination and patient safety through data consistency.

Security and Privacy Measures: As part of integration, FIG. 8 might note that all communications are secured (e.g., using VPN tunnels within the hospital Wi-Fi, TLS encryption for FHIR API calls, etc.). The cobot has its own network credentials and undergoes authentication to access these systems. Audit logs are maintained not just for medication events but also for data access – for example, it logs whenever it queries patient records, ensuring compliance with privacy regulations. Additionally, patient-identifiable information is handled carefully; any cached data on the cobot is protected (encrypted storage) and only retained as long as needed.

Remote Monitoring and Updates: Although not explicitly asked, one can mention that the integration allows remote monitoring of the cobot’s status. For instance, the hospital’s central dashboard could show all active cobots, their locations, battery levels, and recent activities. If FIG. 8 includes an “Administration Console” or similar, that would be an interface where IT or biomedical engineers can check the cobot’s health and push software updates or new drug formulary data. The wireless network link would facilitate such maintenance tasks as well.

In summary, FIG. 8 highlights that the perioperative cobot is fully integrated into the hospital’s digital ecosystem. By syncing with the pharmacy inventory and EMR, it ensures that what it dispenses is always aligned with official records and that those records are updated instantly with each action. Integration via standard protocols like HL7 and FHIR further means the system can be deployed in different hospital IT environments with minimal custom interfacing, leveraging widely used data exchange standards for healthcare. This connectivity also brings efficiency gains (automatic documentation and inventory management) and safety improvements (double-checks against orders, allergy alerts from EMR, etc.). The end result is a cobot that not only physically delivers medications but also communicates every necessary detail to the relevant systems, closing the information loop in perioperative medication management.

Throughout the above sections, various safety and compliance mechanisms have been described in context with specific figures. To summarize and highlight these features collectively:

Tamper-Resistant Secure Storage: The medication compartments remain locked at all times except during an active dispense transaction. The cobot can detect any unauthorized access attempts (e.g., forced entry or if someone tries to pry a drawer). In such a case, it would trigger an alarm and log the event. The compartments are also designed with physical security in mind (sturdy construction, possibly with electronic access logs similar to how ADCs improve medication security).

Multifactor Authentication: By requiring multi-factor credentials for access (badge, biometric, PIN), the system prevents unauthorized users from obtaining medications. Even within the OR team, only those with the right permissions can operate the cobot for dispensing (for example, a housekeeping staff or a student observer in the OR would not have access). This also deters drug diversion and keeps a clear record of responsibility for each dispense.

Authorization Checks (EMR/Scheduling): The integration with patient records and schedules ensures that medications are only dispensed when they align with medical orders and timing. This means, for instance, that a nurse cannot accidentally give a med that wasn’t ordered for that patient, because the cobot would refuse unless an appropriate order is in place. It acts as a real-time guardrail against certain types of medication errors (like administering medication to the wrong patient or at the wrong time).

Contextual Safety Rules: The context-aware system not only helps with efficiency but also with safety – it might warn if a medication is being requested outside of usual context (e.g., a muscle relaxant requested at the end of surgery might prompt “Patient is not intubated – confirm necessity”). These kind of smart prompts can catch potential mistakes (though final decision is with clinicians). Additionally, by anticipating needs, the system can reduce delays in emergencies, which is a patient safety improvement (having epinephrine ready during a code can be lifesaving seconds saved).

Collision Avoidance and Motion Safety: As described, the cobot has multiple layers of obstacle detection to avoid any physical harm or disruption. It operates at safe speeds and uses collaborative-robot safety standards (like ISO 10218 and ISO/TS 15066 guidelines for cobots regarding force and power limiting). An emergency stop is available and the robot will stop if pushed or if it encounters resistance. The path planning avoids collisions by design. These features make it safe to use in the presence of surgical staff who may be moving unpredictably. The sterile field boundary enforcement is another safety aspect to avoid contamination, which indirectly is a patient safety measure (preventing infections).

Logging and Audit Trails: Every action is documented, which is crucial for compliance with healthcare regulations and internal hospital policies. The logs serve as evidence that medications are being handled properly, and they aid in any investigations. For example, if a patient had an adverse reaction, one can check the logs to see exactly what was dispensed, when, and by whom. The audit trail also includes system events (like software updates, restocks, any errors). This thorough record-keeping ensures transparency and accountability.

Fallback and Downtime Procedures: While not explicitly in the figures, the system is designed to fail safe. In the event of a network outage, the cobot can still operate in a limited offline mode using the last synced data (with perhaps some restrictions). If the cobot itself malfunctions, medications can still be retrieved via a manual override by authorized persons (e.g., a physical key or code to open compartments in an emergency). These contingencies ensure that the introduction of the cobot does not create a single point of failure in medication access.

Regulatory Compliance: The entire system is built to meet relevant regulations – for instance, FDA regulations on medical devices, DEA regulations on controlled substances (with features like two-factor auth and inventory checks specifically addressing those requirements), and data security standards for patient information. The design avoids “patent profanity” or absolute language in its programming; instead of hard-coding one way of use, it is configurable to match hospital policy (e.g., whether one-factor or two-factor login is acceptable under certain conditions, how often to force re-authentication, etc.). This flexibility allows it to be compliant in various jurisdictions and adaptable to future rules.

In conclusion, the description above in conjunction with FIGS. 1–8 provides embodiments of the perioperative medication control and dispensation cobot. The system’s hardware components (secure storage, authentication interfaces, mobile base with or without arms, sensors) and software components (control logic for authorization, context-aware decision support, user interface, integration protocols, and safety monitoring) work together to enable a robust solution for delivering medications in the operating room. The described embodiments, including both the humanoid cobot and the alternate wheeled-only version, illustrate how the invention can be implemented in different form factors while achieving the same essential functions. By leveraging multi-factor security, real-time electronic record integration, contextual intelligence, and strict adherence to sterile procedure requirements, this cobot system improves the safety, efficiency, and accountability of perioperative medication management. It will be appreciated by those skilled in the art that various modifications and equivalent arrangements may be made without departing from the scope of the invention, which is defined by the claims that follow. The examples given (such as specific sensor types, communication standards like HL7/FHIR, or particular medication scenarios) are intended to illustrate the enablement of the best modes and are not limiting; other embodiments may employ alternative technologies to achieve similar results in dispensing control, authentication, context inference, user interaction, and system integration as described.

While specific embodiments have been described to enable practice of the disclosed subject matter, they are illustrative and not limiting. Variations, modifications, substitutions, and combinations of the features described can be made by those skilled in the art without departing from the scope of this disclosure. Features described with respect to one embodiment may be used with, or substituted for, features of another. Unless expressly stated otherwise, the terms “comprising,” “including,” and “having” are used in an open, non‑limiting sense; the singular forms “a,” “an,” and “the” include the plural forms; and stated ranges include their endpoints and all subranges. Unless the context clearly indicates otherwise, “and/or” means one or more of the listed items (e.g., “A and/or B” encompasses A alone, B alone, or both A and B); “or” is inclusive (i.e., “A or B” means A, B, or both); and “and” is conjunctive. Headings and ordering are for convenience and do not limit the description. The scope of the claimed subject matter is defined solely by the claims that follow and their legal equivalents.

What is claimed is:

1. An apparatus for perioperative medication control and dispensation, comprising:
a collaborative robotic platform configured for mobile or semi-stationary operation within an operating room environment;
a secure medication storage module on the robotic platform, the storage module comprising multiple access-controlled compartments each adapted to store a different type of medication and configured to dispense a predetermined dosage of the stored medication;
an electronic control unit operatively coupled to the storage module and including a communications interface to hospital systems, the electronic control unit being configured to communicate with a surgical scheduling system and an electronic medical records system to retrieve patient identity, surgical stage, and prescription information for a given patient;
a multi-factor authentication subsystem in communication with the electronic control unit, the authentication subsystem comprising at least one of biometric scanner, a badge reader, and a keypad for personal identification number entry, for verifying identity of an authorized clinician;
a safety sensor system including at least one sterile-field detection sensor and at least one obstacle detection sensor;
a human-machine interaction interface on the robotic platform comprising a touchscreen display and a voice recognition module for clinician input;
wherein the electronic control unit permits dispensing of medication from the storage module only after verifying, based on data retrieved from the surgical scheduling system and the electronic medical records system, that the medication dispensation is authorized for the identified patient in a current surgical stage and after receiving a successful multi-factor authentication of the clinician via the authentication subsystem, and wherein the electronic control unit automatically records an audit log of each dispensation event including the medication type, dosage, time of dispensation, and clinician identity.

2. The apparatus of claim 1, wherein the collaborative robotic platform comprises a motorized base for autonomous mobility and one or more obstacle detection sensors configured to detect and avoid collisions during movement.

3. The apparatus of claim 1, wherein each compartment of the secure medication storage module is designated for a specific medication type and includes a dispensing mechanism configured to release a measured dose of the medication.

4. The apparatus of claim 1, wherein the biometric scanner of the multi-factor authentication subsystem comprises a fingerprint reader configured to scan a clinician’s fingerprint for identity verification.

5. The apparatus of claim 1, wherein the biometric scanner of the multi-factor authentication subsystem comprises a facial recognition camera configured to authenticate the clinician by facial recognition.

6. The apparatus of claim 1, wherein the badge reader of the multi-factor authentication subsystem is configured to scan an identification badge of the clinician to verify clinician credentials.

7. The apparatus of claim 1, wherein the keypad of the multi-factor authentication subsystem is configured to receive entry of a personal identification number code as one of the authentication factors required from the clinician.

8. The apparatus of claim 1, wherein the safety sensor system includes a sterile-field detection sensor, and the electronic control unit is configured to prevent the collaborative robotic platform from breaching a sterile field in the operating room by halting or re-routing movement in response to the sterile-field detection sensor.

9. The apparatus of claim 1, wherein the electronic control unit is further configured to wirelessly transmit inventory update data to a pharmacy inventory management system for each medication dispensed, thereby automatically updating available inventory in real time.

10. The apparatus of claim 1, wherein the electronic control unit is further configured to monitor patient vital sign data or operating room alerts and, upon detecting a predefined critical condition, autonomously initiate preparation of a predetermined medication dose and generate a dispensing prompt requiring clinician confirmation prior to dispensing.

11. A method for controlled perioperative medication dispensation using a collaborative robotic platform, the method comprising:
receiving, via a user interface of the collaborative robotic platform, a request from a clinician to dispense a specified medication for a patient;
retrieving patient identity, surgical stage, and medication order information for the patient from at least one of a surgical scheduling system and an electronic medical records system;
verifying, based on the retrieved patient identity, surgical stage, and medication order information, that the requested medication is authorized for administration to the patient during a current stage of a surgical procedure;
obtaining multi-factor authentication of the clinician requesting the medication by requiring at least two distinct forms of identification from the clinician;
dispensing, by the collaborative robotic platform, a measured dose of the authorized medication from the secure medication storage module upon successful verification and authentication;
recording, in an audit log, data associated with the dispensation event, including at least the medication dispensed, the dispensed dosage, the time of dispensation, the clinician’s identity, and the location of the dispensation.

12. The method of claim 11, wherein obtaining multi-factor authentication of the clinician comprises scanning the clinician’s fingerprint using a fingerprint reader on the collaborative robotic platform.

13. The method of claim 11, wherein obtaining multi-factor authentication of the clinician comprises capturing an image of the clinician’s face and performing facial recognition to verify the clinician’s identity.

14. The method of claim 11, wherein obtaining multi-factor authentication of the clinician comprises reading an identification badge of the clinician to verify clinician credentials prior to dispensing.

15. The method of claim 11, wherein obtaining multi-factor authentication of the clinician comprises receiving entry of a personal identification number from the clinician via the user interface.

16. The method of claim 11, further comprising monitoring patient vital signs and operating room status in real time, and upon detecting a predefined critical condition, automatically presenting a recommended medication for dispensation via the collaborative robotic platform and requiring a confirmation input from a clinician before dispensing the recommended medication.

17. A perioperative medication management system, comprising:
a collaborative robotic medication dispensing apparatus configured to store and dispense medications within an operating room environment;
a surgical scheduling system storing scheduling data for surgical procedures;
an electronic medical records system storing patient data and medication orders;
a communication network coupling the robotic medication dispensing apparatus with the surgical
scheduling system and the electronic medical records system;
wherein the robotic medication dispensing apparatus is configured to retrieve patient-specific surgical information from the surgical scheduling system and medication order information from the electronic medical records system via the communication network, to verify that a medication dispensation is authorized for a particular patient based on the retrieved information, to require successful multi-factor authentication of a clinician before permitting dispensation, and to dispense the medication upon verification of authorization and clinician authentication.

18. The system of claim 17, further comprising a pharmacy inventory management system communicatively connected to the robotic medication dispensing apparatus, wherein the robotic medication dispensing apparatus is configured to transmit inventory usage data to the pharmacy inventory management system for each medication dispensed to update medication inventory levels in real time.

19. The system of claim 17, further comprising a patient vital sign monitoring system that provides real-time vital sign data of the patient to the robotic medication dispensing apparatus, wherein the robotic medication dispensing apparatus is configured to detect a predefined critical patient condition from the vital sign data and automatically generate a medication dispensing alert or recommendation in response to the detected condition.

20. The system of claim 17, wherein the robotic medication dispensing apparatus is configured to transmit, via the communication network, a record of each medication dispensation event to the electronic medical records system to document the dispensation in the patient’s medical record in real time.
